(19) United States
(12) Patent Application Publication
(10) Pub. No.: US 2005/0020854 A1
(43) Pub. Date: Jan. 27, 2005

(54) Alkyl(en)ylglycerinether carboxylic acids

(76) Inventors: Ansgar Behler, Bottrop (DE); Thomas Albers, Duesseldorf (DE); Karl Heinz Schmid, Mettmann (DE)

Correspondence Address: COGNIS CORPORATION;PATENT DEPARTMENT, 300 BROOKSIDE AVENUE, AMBLER, PA, 19002, US

(21) Appl. No.: 10/495,016

(22) PCT Filed: Oct. 31, 2002
(86) PCT No.: PCT/EP02/12146

(30) Foreign Application Priority Data
Nov. 9, 2001 (DE) 101 55 095.2

(51) Int. Cl. C07C 59/125; C07C 59/305

(52) U.S. Cl. 562/583

(57) ABSTRACT

Alk(en)yl glycerol ether carboxylic acids which are prepared by reacting, in the presence of alkali, (a) an alk(en)yl glycerol ether compound of the general formula (I):
R¹O(A)ₙCH₂CH[(A)ₘOR²]CH₂(A)ₚOH (I)
wherein R¹ and R² each independently represents a hydrogen or an alk(en)yl group having from 4 to 22 carbon atoms, with the proviso that at least one of R¹ and R² represents an alk(en)yl group having from 4 to 22 carbon atoms, each A independently represents a C₂H₄O and/or C₃H₆O group and n, m and p each independently represents a number of from 0 to 10; and (b) an α-halocarboxylic acid of the general formula (II):
XCR³R⁴COOH (II)
wherein R³ represents a hydrogen or a —CH₃ group, R⁴ represents a hydrogen or an alk(en)yl group having from 1 to 6 carbon atoms and X represents a halogen; are described along with various uses for the same.

### FIELD OF THE INVENTION

The invention relates to alk(en)yl glycerol ether carboxylic acids, to a process for their production and to their use as cleaning and foaming agents and as emulsifiers.

### PRIOR ART

Besides a number of other ingredients, surface-active preparations, such as for example dishwashing detergents, laundry detergents and cleaners, and cosmetic preparations contain anionic surfactants as their most important component, the anionic surfactants being responsible not only for the cleaning effect, but also for the generation of foam. Accordingly, there is still a need on the market for new anionic surfactants which, besides their cleaning and foaming effect, also have other positive properties, such as good mucous membrane compatibility for example, and which, in addition, can be produced relatively inexpensively, simply and in salt-free form.

Accordingly, the problem addressed by the present invention was to provide new anionic surfactants, which would have the above-mentioned properties and which could be produced inexpensively, simply and in salt-free form, and a process for their production.

### DESCRIPTION OF THE INVENTION

The present invention relates to alk(en)yl glycerol ether carboxylic acids obtainable by reacting mono- and/or dialk(en)yl glycerol ethers and addition products thereof with ethylene oxide and/or propylene oxide corresponding to formula (I):
R¹O(A)ₙCH₂CH[(A)ₘOR²]CH₂(A)ₚOH (I)
in which R¹ and R² independently of one another represent H or a linear and/or branched alkyl and/or alkenyl group containing 4 to 22 carbon atoms, but at least one of the substituents R¹ and R² is a linear and/or branched alkyl and/or alkenyl group containing 4 to 22 carbon atoms, A is a C₂H₄O and/or C₃H₆O group and n, m and p independently of one another stand for 0 or a number of 0.5 to 10,
with α-halocarboxylic acids corresponding to formula (II):
XCR³R⁴COOH (II)
in which R³ is H or a CH₃ group, R⁴ is H or a linear and/or branched alkyl and/or alkenyl group containing 1 to 6 carbon atoms and X is halogen, in the presence of alkali.

The present invention also relates to a process for the production of alk(en)yl glycerol ether carboxylic acids, in which mono- and/or dialk(en)yl glycerol ethers and addition products thereof with ethylene oxide and/or propylene oxide corresponding to formula (I):
R¹O(A)ₙCH₂CH[(A)ₘOR²]CH₂(A)ₚOH (I)
in which R¹ and R² independently of one another represent H or a linear and/or branched alkyl and/or alkenyl group containing 4 to 22 carbon atoms, but at least one of the substituents R¹ and R² is a linear and/or branched alkyl and/or alkenyl group containing 4 to 22 carbon atoms, A is a C₂H₄O and/or C₃H₆O group and n, m and p independently of one another stand for 0 or a number of 0.5 to 10,
are reacted with α-halocarboxylic acids corresponding to formula (II):
XCR³R⁴COOH (II)
in which R³ is H or a CH₃ group, R⁴ is H or a linear and/or branched alkyl and/or alkenyl group containing 1 to 6 carbon atoms and X is halogen, preferably chloride,
in the presence of alkali.

It has surprisingly been found that alk(en)yl glycerol ether carboxylic acids show good foaming behavior and a good cleaning effect and, besides their surface-active properties, good emulsifying properties. Accordingly, these compounds may be used in a number of surface-active preparations in which the presence of anionic surfactants or emulsifiers is desirable, for example in laundry detergents, dishwashing detergents and cleaners and also cosmetic emulsions. At the same time, they are dermatologically safe and can be prepared simply by reacting mono- and/or dialk(en)yl glycerol ethers and halocarboxylic acids in the presence of alkali. It is a particular advantage that these compounds can be obtained in substantially salt-free form so that troublesome effects attributable to the presence of salt ions can be avoided.

Ethoxylated and/or propoxylated alk(en)yl glycerol ether carboxylic acids show extremely good foaming behavior.

Alk(en)yl glycerol ether carboxylic acids

The present invention relates to alk(en)yl glycerol ether carboxylic acids [alk(en)yl=alkyl and/or alkenyl] obtainable by reacting mono- and/or dialk(en)yl glycerol ethers and addition products thereof with ethylene oxide and/or propylene oxide corresponding to formula (I) and α-halocarboxylic acids corresponding to formula (II). In these formulae, R¹ and R² independently of one another represent H or a linear and/or branched alkyl and/or alkenyl group containing 4 to 22, preferably 8 to 18 and more particularly 12 to 18 carbon atoms, R³ is H or a CH₃ group, R⁴ is H or a linear and/or branched alkyl and/or alkenyl group containing 1 to 6 and preferably 2 to 4 carbon atoms, A is a C₂H₄O or C₃H₆O group and n, m and p independently of one another stand for 0 or a number of 0.5 to 10. Where A is a C₃H₆O group, n, m and p preferably independently of one another stand for numbers of 0 to 5.

One embodiment of the present invention is characterized by the use of mono- and/or dialk(en)yl glycerol ethers corresponding to formula (I), in which R¹ and R² represent a linear and/or branched alkyl and/or alkenyl group containing 4 to 22, preferably 8 to 18 and more particularly 12 to 18 carbon atoms and n, m and p stand for 0 or numbers of 0.5 to 10.

Another embodiment of the present invention is characterized by the use of mono- and/or dialk(en)yl glycerol ethers corresponding to formula (I), in which (a) R¹ is H and R² represents a linear and/or branched alkyl and/or alkenyl group containing 4 to 22, preferably 8 to 18 and more particularly 12 to 18 carbon atoms or (b) R² is H and R¹ represents a linear and/or branched alkyl and/or alkenyl group containing 4 to 22, preferably 8 to 18 and more particularly 12 to 18 carbon atoms and n, m and p stand for 0.

A particularly preferred embodiment of the present invention is characterized by the use of mono- and/or dialk(en)yl glycerol ethers corresponding to formula (I), in which (a) R¹ and R² represent a linear and/or branched alkyl and/or alkenyl group containing 4 to 22, preferably 8 to 18 and more particularly 12 to 18 carbon atoms and n and m stand for 0 or a number of 0.5 to 5, more particularly 0, and p is a number of 0.5 to 10, more particularly 2 to 6. Ethoxylated compounds, i.e. those in which A is a C₂H₄O group, are most particularly preferred.

Another particularly preferred embodiment of the present invention is characterized by the use of mono- and/or dialk(en)yl glycerol ethers corresponding to formula (I), in which (a) R¹ is a linear and/or branched alkyl and/or alkenyl group containing 4 to 22, preferably 8 to 18 and more particularly 12 to 18 carbon atoms and R² is H and n stands for 0 or a number of 0.5 to 5 and m and p independently of one another stand for a number of 0.5 to 10, more particularly 1 to 10, the sum of m+p preferably being in the range from 1 to 15 and more particularly 2 to 10. Ethoxylated compounds (A=C₂H₄O group) are particularly preferred.

Another particularly preferred embodiment of the invention is characterized by the use of mono- and/or dialk(en)yl glycerol ethers corresponding to formula (I), in which (b) R² represents a linear and/or branched alkyl and/or alkenyl group containing 4 to 22, preferably 8 to 18 and more particularly 12 to 18 carbon atoms and R¹ is H and m stands for 0 or a number of 0.5 to 10 and more particularly 1 to 10, the sum of n+p preferably being in the range from 1 to 15 and more particularly 2 to 10. Ethoxylated compounds (A=C₂H₄O group) are again particularly preferred.

α-Halocarboxylic acids of formula (II), in which R³ and R⁴ represent H and X is halogen, are preferably used.

Mono-C₁₂-glycerol ether carboxylic acids, mono-C₁₆/C₁₈18:1-glycerol ether carboxylic acids (optionally unsaturated), mono-C₁₂-glycerol ether carboxylic acid ethoxylated with 4 mol ethylene oxide (p=4EO) are particularly preferred.

Accordingly, both mono- and dialk(en)yl glycerol ether carboxylic acids are suitable for the purposes of the invention, monoalk(en)yl glycerol ether carboxylic acids being preferred and ethoxylated monolauryl glycerol ether carboxylic acids being particularly preferred. The alkali metal, alkaline earth metal and amine salts of the alk(en)yl glycerol ether carboxylic acids are also included in the scope of the invention.

The alk(en)yl glycerol ether carboxylic acids according to the invention are used in surface-active preparations, preferably in laundry detergents, dishwashing detergents and cleaners, and cosmetic and/or pharmaceutical preparations in quantities of 0.01 to 60, preferably 0.05 to 30 and more particularly 2.5 to 20% by weight, based on the active substance content.

Production of alk(en)yl glycerol ether carboxylic acids

The alk(en)yl glycerol ether carboxylic acids according to the invention are obtained by reaction of mono- and/or dialk(en)yl glycerol ethers corresponding to formula (I) with α-halocarboxylic acids corresponding to formula (II) in the presence of alkali. The alk(en)yl glycerol ether carboxylic acids are obtained by addition of acids. The preferred alk(en)yl glycerol ether carboxylic acids obtainable by this reaction were mentioned in the previous chapter.

To carry out the reaction, the mono- and/or dialk(en)yl glycerol ether (prepared, for example, in accordance with DE 4118568 A1 by reaction of glycerol with alk(en)yl sulfate, sodium salts in an alkaline medium) or addition products thereof with ethylene oxide and/or propylene oxide [formula (I)] is/are heated with the α-halocarboxylic acid corresponding to formula (II) in a molar ratio of 1:4 to 1:3 and preferably 1:1.0 to 1:1.6 to temperatures of 70 to 95° C. and preferably to temperatures of 89 to 90° C. in the reaction vessel. 1.2 to 1.6 mol solid alkali, preferably alkali metal hydroxide, for example in the form of NaOH microprills, per mol mono- and/or dialk(en)yl glycerol ether are then added in several portions over a period of 3 to 6 and preferably 4 to 5 hours at that temperature, followed by stirring for another 1 to 3 hours. To release the free acid, the reaction mixture is diluted while stirring with water to an active substance content of 20 to 60 and preferably 25 to 40% by weight and acidified with 10 to 96 and preferably 20% acid, preferably sulfuric acid, to a pH of 1 to 4 and preferably 2 to 3.5 (measured as 10%). The resulting phase separation of the organic and aqueous phases was improved by addition of 5% isopropanol. The organic phase was removed and the reaction product was dried in vacuo in a rotary evaporator.

Where addition products with ethylene oxide and/or propylene oxide of the mono- and/or dialk(en)yl glycerol ethers corresponding to formula (I) are used, they are obtained in known manner by ethoxylation and/or propoxylation of the mono- and/or dialk(en)yl glycerol ethers, a narrow or broad homolog distribution being obtained according to the catalyst used. Naturally only the free hydroxyl groups are ethoxylated and/or propoxylated. Where R¹ or R² represents one of the defined alkyl and/or alkenyl groups and n or m is a number of 0.5 to 10, the glycerol has to be ethoxylated and/or propoxylated in known manner before the production of the mono- and/or dialk(en)yl glycerol ethers.

### COMMERCIAL APPLICATIONS

The alk(en)yl glycerol ether carboxylic acids according to the invention may be adjusted to any concentration by addition of water; their water content may be in the range from 20 to 85% by weight and is preferably in the range from 25 to 60% by weight and more particularly in the range from 30 to 40% by weight.

The alk(en)yl glycerol ether carboxylic acids or their salts may be used as surfactants in surface-active preparations. Surface-active preparations in the context of the invention are, preferably, laundry detergents, dishwashing detergents and cleaners and cosmetic and/or pharmaceutical preparations, more particularly cosmetic and/or pharmaceutical preparations. These surface-active preparations may contain further auxiliaries and additives selected from pearlizing waxes, consistency factors, thickeners, superfatting agents, stabilizers, silicone compounds, fats, waxes, antioxidants, antidandruff agents, swelling agents, tyrosine inhibitors, hydrotropes, solubilizers, preservatives, perfume oils, dyes, other surfactants and other ingredients typical, for example, of laundry detergents, dishwashing detergents and cleaners. Cosmetic and/or pharmaceutical preparations are, preferably, oral hygiene and dental care preparations, hair shampoos, hair lotions, foam baths, shower baths, creams, lotions, gels, emulsions, wax/fat compounds, stick preparations or ointments. Besides the alk(en)yl glycerol ether carboxylic acids according to the invention, these surface-active preparations may contain other known ingredients typical of the particular application in the usual concentrations.

The compounds according to the invention show not only cleaning properties, but also foaming properties. In addition, these alk(en)yl glycerol ether carboxylic acids, preferably the C_{16/18} glycerol ether carboxylic acids, have emulsifying properties and may therefore be used in cosmetic and/or pharmaceutical preparations. Accordingly, the present invention also relates to the use of the alk(en)yl glycerol ether carboxylic acids according to the invention as cleaning and/or foaming components and as emulsifiers. More particularly, C₁₆₋₁₈ glycerol ether carboxylic acids are used as emulsifiers in all types of emulsion known to the expert.

Typical cosmetic and/or pharmaceutical cleaning preparations preferably have the following composition, based on their active substance content:
(a) 0.05 to 20, preferably 0.5 to 10 and more particularly 2.5 to 18% by weight alk(en)yl glycerol ether carboxylic acids,
(b) 0.05 to 15, preferably 0.5 to 10 and more particularly 2.5 to 7.5% by weight betaines and optionally
(c) 0 to 15, preferably 0.5 to 10 and more particularly 2.5 to 7.5% by weight other anionic surfactants,
   with the proviso that the quantities shown add up to 100% by weight, optionally with water and/or other auxiliaries and additives.

Typical liquid laundry detergents, dishwashing detergents and cleaners preferably have the following composition, based on their active substance content:
(a) 2.5 to 30, preferably 7 to 25 and more particularly 10 to 20% by weight alk(en)yl glycerol ether carboxylic acids,
(b) 0.05 to 15, preferably 0.5 to 10 and more particularly 2.5 to 7.5% by weight betaines and optionally
(c) 2.5 to 30, preferably 7 to 25 and more particularly 10 to 20% by weight other anionic surfactants,
   with the proviso that the quantities shown add up to 100% by weight, optionally with water and/or other auxiliaries and additives.

Typical cosmetic and/or pharmaceutical emulsions preferably have the following composition, based on their active substance content:
(a) 0.05 to 15, preferably 0.5 to 10 and more particularly 1 to 5% by weight alk(en)yl glycerol ether carboxylic acids and preferably C_{16/18} glycerol ether carboxylic acids,
(b) 3 to 30, preferably 5 to 20 and more particularly 7 to 15% by weight oil components and optionally
(c) 0.5 to 20 and preferably 2.5 to 10% by weight consistency factors,
   with the proviso that the quantities shown add up to 100% by weight with water and optionally other auxiliaries and additives.

### EXAMPLES

The following Examples are intended to illustrate the invention without limiting it any way.

### Example 1

Preparation of mono-C₁₂-glycerol ether carboxylic acid

In a reaction vessel, 260 g mono-C₁₂-glycerol ether (1 mol) and 163.1 g (1.4 mol) sodium monochloroacetate were heated to a temperature of 60 to 80° C. 56.0 g (1.4 mol) sodium hydroxide microprills were then added in portions with mechanical stirring over a period of 4 hours. Following an after-reaction time of 1.5 hours and after the theoretical quantities of chloride had been released (10.4% Cl⁻, as determined to DGF-H-III 9), the reaction was terminated. The reaction product was diluted with 114.1 g water to an active substance content of ca. 60% by weight (A).

To release the free acid, 498.1 g of this product were then diluted with another 250 g water (corresponding to an active substance content of ca. 40% by weight) and acidified with 182 g 20% sulfuric acid. In order to improve phase separation, 50 ml isopropanol were also added, the organic phase was then separated from the water phase and the ether carboxylic acid was dried in vacuo in a rotary evaporator (ca. 20 mbar). A yellow clear liquid was obtained as the reaction product (B).

| | Carboxylate, | Ether carboxylic |
|---|---|---|
| | sodium salt (A) | acid (B) |
| Dry residue: | 56.9 | |
| Inorganic chloride: | 6.3 | 0.08 |
| Glycolic acid: | 3.3 | 0.6 |
| Diglycolic acid | 1.3 | 0.2 |
| Residual C₁₂ glycerol ether | | 8.6 |

### Example 2

Preparation of mono-C₁₂-glycerol ether+4EO ether carboxylic acid

In a reaction vessel, 436.0 g mono-C₁₂-glycerol ether (1 mol) and 122.3 g (1.05 mol) sodium monochloroacetate were heated to a temperature of 60° C. 42.0 g (1.05 mol) sodium hydroxide microprills were then added in portions with mechanical stirring over a period of 4 hours. Following an after-reaction time of 1.75 hours and after the theoretical quantities of chloride had been released (6.2% Cl, as determined to DGF-H-ll 9), the reaction was terminated.(A).

To release the free acid, 297.0 g of this product were then diluted with 148.5 g water (corresponding to an active substance content of ca. 54% by weight) and acidified with 104.5 g 20% sulfuric acid. The organic phase was separated from the water phase and the ether carboxylic acid was dried in vacuo in a rotary evaporator (ca. 20 mbar). A yellow clear liquid was obtained as the reaction product (B).

| | Carboxylate, | Ether carboxylic |
|---|---|---|
| | sodium salt (A) | acid (B) |
| Water | | 0.5% |
| Inorganic chloride: | 6.1% | 0.07% |
| Glycolic acid: | 1.4% | |
| Diglycolic acid | 0.9% | |
| OH value | 85 | 84 |
| Acid value | −0.1 | 75 |

### Example 3

Preparation of mono-C_{16/18}-glycerol ether carboxylic acid

In a reaction vessel, 374.3 g mono-C_{16/18}-glycerol ether (1.1 mol) and 134.5 g (1.155 mol) sodium monochloroacetate were heated to a temperature of 85° C. 46.2 g (1.155 mol) sodium hydroxide microprills were then added in portions with mechanical stirring over a period of 4 hours. Following an after-reaction time of 3.5 hours and after the theoretical quantities of chloride had been released (7.3% Cl, as determined to DGF-H-III 9), the reaction was terminated (A).

To release the free acid, 304.0 g of this product were then diluted with 152.0 g water (corresponding to an active substance content of ca. 54% by weight) and acidified with 107.0 g 20% sulfuric acid. The organic phase was separated from the water phase and the ether carboxylic acid was dried in vacuo in a rotary evaporator (ca. 20 mbar). A beige solid was obtained as the reaction product (B).

| | Carboxylate, | Ether carboxylic |
|---|---|---|
| | sodium salt (A) | acid (B) |
| Water | | 0.2% |
| Inorganic chloride: | 7.3 | 0% |
| OH value | 119 | 95 |
| Acid value | 0 | 27 |

Performance Tests

### Example 4

To study foaming behavior, an aqueous solution of the mono-C₁₂-glycerol ether carboxylate produced in accordance with Example 1 was prepared and the foam volume was determined to DIN Standard EN 12728, 01/00 (0.1 g active substance/l; 40° C.; 0° dH; pH 6.0). In this method, the foam is produced by beating the liquid sample for 30 seconds in a gas jar with a horizontally aligned perforated plate attached to a handle. The foam volume generated is measured immediately after the end of beating and 5, 10 and 20 minutes thereafter.

TABLE 1
Foam measurements of the aqueous surfactant solution of Example 1

| | |
|---|---|
| Foam height [ml] | 1 |
| Immediately | 190 |
| After 5 mins. | 190 |
| After 10 mins. | 180 |
| After 20 mins. | 180 |

### Example 5

TABLE 2
rotor foam; 0° dH; 40° C.; pH 6; 0.5 g/l

| | C₁₂ monoglycerol ether | C₁₂ monoglycerol ether + 4EO |
|---|---|---|
| | carboxylate | carboxylate |
| 30 s | 110 ml | 280 ml |
| 60 s | 150 ml | 450 ml |
| 90 s | 180 ml | 680 ml |
| 120 s | 205 ml | 850 ml |

### Example 6

TABLE 3
rotor foam; 16° dH; 40° C.; pH 6; 0.5 g/l

| | |
|---|---|
| C₁₂ monoglycerol ether | C₁₂ Monoglycerol ether + 4EO |
| carboxylate | carboxylate |

| | | |
|---|---|---|
| 30 s | 80 ml | 220 ml |
| 60 s | 90 ml | 350 ml |
| 90 s | 90 ml | 470 ml |
| 120 s | 90 ml | 520 ml |

### Example 7

Mucous membrane compatibility was evaluated by the in vitro HET-CAM test using the reaction time method (conc. 5% in 2×-distilled water, pH=6). The results are set out in Table 2.

TABLE 4
Mucous membrane compatibility of the aqueous surfactant solution

| | Irritation | Standard | |
|---|---|---|---|
| Substance | value Q | deviation | Evaluation |
| C₁₂ monoglycerol ether | 0.76 | 0.03 | Slightly irritating |
| carboxylic acid | | | |
| C₁₂ monoglycerol ether + | 0.00 | | Slightly irritating |
| 4EO carboxylic acid | | | |
| Texapon ASV | 1.0 | 0.04 | Moderately |
| Sodium Laureth Sulfate | | | irritating |

1-11. (Canceled)

12. An alk(en)yl glycerol ether carboxylic acid prepared by a process comprising reacting, in the presence of alkali:
(a) an alk(en)yl glycerol ether compound of the general formula (I):
R¹O(A)ₙCH₂CH[(A)ₘOR²]CH₂(A)ₚOH (I)
wherein R¹ and R² each independently represents a hydrogen or an alk(en)yl group having from 4 to 22 carbon atoms, with the proviso that at least one of R¹ and R² represents an alk(en)yl group having from 4 to 22 carbon atoms, each a independently represents a C₂H₄O and/or C₃H₆O group and n, m and p each independently represents a number of from 0 to 10; and
(b) an α-halocarboxylic acid of the general formula (II):
XCR³R⁴COOH (II)
wherein R³ represents a hydrogen or a —CH₃ group, R⁴ represents a hydrogen or an alk(en)yl group having from 1 to 6 carbon atoms and X represents a halogen.

13. The alk(en)yl glycerol ether carboxylic acid according to claim 12, wherein R¹ and R² both independently represent an alk(en)yl group having from 4 to 22 carbon atoms.

14. The alk(en)yl glycerol ether carboxylic acid according to claim 12, wherein one of R¹ and R² represents an alk(en)yl group having from 4 to 22 carbon atoms and the other represents a hydrogen, and wherein n, m and p each represents zero.

15. The alk(en)yl glycerol ether carboxylic acid according to claim 12, wherein at least one of R¹ and R² represents an alk(en)yl group having from 8 to 18 carbon atoms.

16. The alk(en)yl glycerol ether carboxylic acid according to claim 12, wherein at least one of R¹ and R² represents an alk(en)yl group having from 12 to 18 carbon atoms.

17. The alk(en)yl glycerol ether carboxylic acid according to claim 12, wherein R¹ represents hydrogen and R² represents an alk(en)yl group having from 4 to 22 carbon atoms, and wherein m represents a number of from 0 to 5 and n and p each represents a number of from 0.5 to 10.

18. The alk(en)yl glycerol ether carboxylic acid according to claim 12, wherein R² represents hydrogen and R¹ represents an alk(en)yl group having from 4 to 22 carbon atoms, and wherein n represents a number of from 0 to 5 and m and p each represents a number of from 0.5 to 10.

19. The alk(en)yl glycerol ether carboxylic acid according to claim 12, wherein R³ and R⁴ each represent hydrogen.

20. The alk(en)yl glycerol ether carboxylic acid according to claim 19, wherein at least one of R¹ and R² represents an alk(en)yl group having from 8 to 18 carbon atoms.

21. The alk(en)yl glycerol ether carboxylic acid according to claim 19, wherein at least one of R¹ and R² represents an alk(en)yl group having from 12 to 18 carbon atoms.

22. A process for preparing an alk(en)yl glycerol ether carboxylic acid, said process comprising reacting, in the presence of alkali:
(a) an alk(en)yl glycerol ether compound of the general formula (I):
R¹O(A)ₙCH₂CH[(A)ₘOR²]CH₂(A)ₚOH (I)
wherein R¹ and R² each independently represents a hydrogen or an alk(en)yl group having from 4 to 22 carbon atoms, with the proviso that at least one of R¹ and R² represents an alk(en)yl group having from 4 to 22 carbon atoms, each A independently represents a C₂H₄₀ and/or C₃H₆O group and n, m and p each independently represents a number of from 0 to 10; and
(b) an α-halocarboxylic acid of the general formula (II):
XCR³R⁴COOH (II)
wherein R³ represents a hydrogen or a —CH₃ group, R⁴ represents a hydrogen or an alk(en)yl group having from 1 to 6 carbon atoms and X represents a halogen.

23. The process according to claim 22, wherein R¹ and R² both independently represent an alk(en)yl group having from 4 to 22 carbon atoms.

24. The process according to claim 22, wherein one of R¹ and R² represents an alk(en)yl group having from 4 to 22 carbon atoms and the other represents a hydrogen, and wherein n, m and p each represents zero.

25. The process according to claim 22, wherein at least one of R¹ and R² represents an alk(en)yl group having from 8 to 18 carbon atoms.

26. The process according to claim 22, wherein at least one of R¹ and R² represents an alk(en)yl group having from 12 to 18 carbon atoms.

27. The process according to claim 22, wherein R¹ represents hydrogen and R² represents an alk(en)yl group having from 4 to 22 carbon atoms, and wherein m represents a number of from 0 to 5 and n and p each represents a number of from 0.5 to 10.

28. The process according to claim 22, wherein R2 represents hydrogen and R¹ represents an alk(en)yl group having from 4 to 22 carbon atoms, and wherein n represents a number of from 0 to 5 and m and p each represents a number of from 0.5 to 10.

29. The process according to claim 22, wherein R³ and R⁴ each represent hydrogen.

30. A composition comprising an alk(en)yl glycerol ether carboxylic acid according to claim 12 in an amount of from 0.01 to 60% by weight based on active substance content.
